(19) United States
(12) Patent Application Publication
(10) Pub. No.: US 2006/0239986 A1
(43) Pub. Date: Oct. 26, 2006

(54) Method for the formation of hydrogel multilayers through surface initiated photopolymerization

(76) Inventors: Victor Hugo Perez-Luna, Naperville, IL (US); Fouad A. Teymour, Mount Prospect, IL (US); Seda Kizilel, Chicago, IL (US)

Correspondence Address: BELL, BOYD & LLOYD, LLC, PO BOX 1135, CHICAGO, IL, 60690-1135, US

(21) Appl. No.: 11/340,120

(22) Filed: Jan. 26, 2006

Related U.S. Application Data
(60) Provisional application No. 60/647,667, filed on Jan. 26, 2005.

(51) Int. Cl. A61K 38/43; A61K 9/14

(52) U.S. Cl. 424/94.1; 424/486

(57) ABSTRACT

Multiple-layer hydrogels and methods of forming hydrogels having multiple layers are disclosed. The hydrogels are formed of prepolymerization solutions comprising monomers or macromers, particularly polyethylene glycol. The layers are covalently bonded, and the hydrogels may comprise two or more layers.

### PRIORITY CLAIM

This patent claims priority to U.S. Provisional Patent Application No. 60/647,667, titled “Method for the Formation of Hydrogel Multilayers Through Surface Initiated Photopolymerization,” filed Jan. 26, 2005, the contents of which are hereby fully incorporated by reference herein.

### BACKGROUND OF THE INVENTION

The present invention relates generally to hydrogels. More specifically, the present invention relates to multiple-layer hydrogels and methods of forming hydrogels having multiple layers.

Hydrogels have been used for many biomedical applications, such as drug delivery and as biomaterials. Hydrogels are particularly useful in such applications because of their high water content, transport properties, and tissue-like physical and mechanical behaviors. Other biomedical applications include using hydrogels as barriers between drugs or devices and living tissue. Hydrogels can be used to encapsulate biological materials and have also been used in the development of electrochemical and optical sensors. Additionally, hydrogels may also be used with trans-dermal photopolymerization for cartilage tissue engineering applications.

Of particular use in the industry, degradable polyethylene glycol (“PEG”) hydrogels can be formed in-situ under mild conditions. These PEG hydrogels have been found particularly useful as drug carriers for the release of proteins and low molecular weight drugs, as well as in wound coverings. Moreover, because PEG hydrogels are biocompatible and semipermeable, they have been used as barriers in tissue engineering, or therapeutic cell transplantation, to prevent the rejection of transplanted cells by a host's immune system.

Traditional applications involving drug delivery, tissue barriers or cell encapsulation rely on the formation of single hydrogel layers. Single-layer hydrogels consist of a plain membrane, which is formed during a single procedural step. However, the composition and properties of single membranes can limit the potential applications of hydrogels. This is because it is often difficult to find a single hydrogel material with the desired properties for a particular application, as a specific application demands properties that cannot be met within a single layer. Accordingly, it is necessary to provide hydrogels that allow multiple properties for use in multiple applications.

### SUMMARY OF THE INVENTION

The present invention provides methods and products for formation of hydrogel multilayers. A hydrogel can be formed using various monomers or macromers. Although the following focuses on using PEG hydrogels as a model system, it should be appreciated that other monomers or macromers can be used to form the multilayer hydrogels.

The present invention provides in an embodiment a method for formation of hydrogel multilayers. The method begins with preparing a first layer of hydrogel on a solid support, such as a glass slide. First, a prepolymerization solution is mixed. The solid support is derivatized with a photoinitiator, such as eosin, and then the prepolymerization solution is deposited onto the solid support. The solution on the solid support is then exposed to radiation, forming the first hydrogel layer.

In an embodiment, the radiation used to form the first hydrogel layer has a wavelength of 514 nanometers.

In an embodiment, the first hydrogel layer is then exposed to the photoinitiator, causing covalent attachment of amine groups in the hydrogel with the carboxyl groups present in the photoinitiator. Prepolymerization solution is deposited onto the first layer and exposed to radiation, thereby attaching the second hydrogel layer to the first hydrogel layer.

In an embodiment, the radiation used to form the second hydrogel layer has a wavelength of 514 nanometers.

In an embodiment, the process may be used to form a third, or more, hydrogel layers.

The components of the solutions and the pattern of the radiation may be varied during the formation of any layer, yielding multilayer hydrogels with varied functionality.

In a further embodiment, hydrogels can also incorporate cells, pharmaceutical agents or other biologically active materials within the layers.

In an embodiment described herein, homogenous monomers are used to form a hydrogel.

Another embodiment includes using a mixture of more than one type of monomer to form a hydrogel.

In a further embodiment, one or more types of macromers are utilized during the formation of a hydrogel. It should be appreciated that other monomers or macromers, in addition to the model system shown, may be used to perform the formation of hydrogel multilayers.

In yet a further embodiment, hydrogel multilayers are sequentially formed of PEG on eosin derivatized surfaces.

In another embodiment, complex features on the surface of the hydrogel layers may be formed.

In yet another embodiment, three dimensional (3-D) patterns may be formed on the hydrogel layers.

Additional features and advantages of the present invention are described in, and will be apparent from, the following Detailed Description of the Invention and the figures.

### BRIEF DESCRIPTION OF THE FIGURES

FIG. 1 is a schematic representation illustrating coupling of cysteamine to PEG diacrylate by a conjugate addition reaction according to an embodiment of the present invention.

FIG. 2 is a schematic representation of multilayer hydrogel formation according to an embodiment of the present invention.

FIG. 3 is a graphic representation of a single layer hydrogel according to an embodiment of the present invention.

FIG. 4 is a graphic representation of a double layer hydrogel according to an embodiment of the present invention.

FIG. 5a and FIG. 5b are graphic representations of a multilayer hydrogel according to an embodiment of the present invention.

FIG. 6 is a schematic representation of multidimensional hydrogel layers according to an embodiment of the present invention.

FIG. 7 is a schematic representation of multidimensional hydrogel layers according to another embodiment of the present invention.

### DETAILED DESCRIPTION OF THE INVENTION

The present invention provides multilayer hydrogels. In the embodiment described herein, a first layer of hydrogel is generated on a support, and further layers are added to the first layer. In an embodiment the hydrogel layers are made up of homogeneous monomers, but it will be appreciated that any suitable monomer or macromer may be used to form the hydrogel.

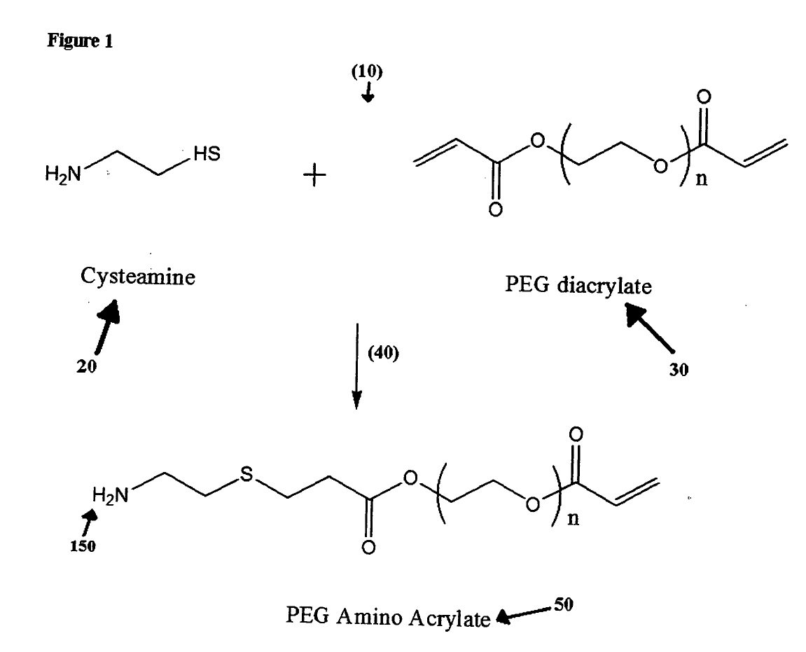

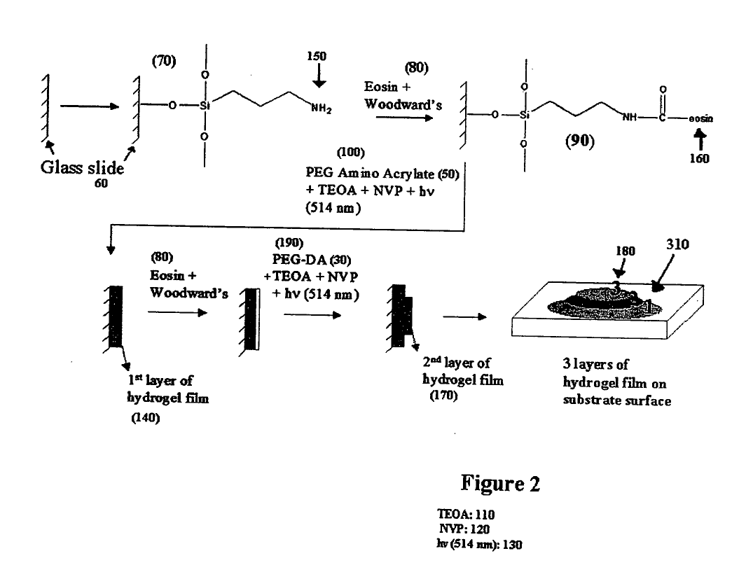

Referring now to the drawings, and in particular to FIG. 1, a schematic representation of one step in preparation of the multilayer hydrogels of the present invention is shown. In particular, FIG. 1 illustrates preparation of a precursor to a prepolymerization solution, in this case including PEG amino acrylate 50. The PEG amino acrylate 50 is formed through coupling of cysteamine 20 to PEG diacrylate 30. In particular, a PEG diacrylate monomer 30 is modified through a conjugate addition reaction 40, also known as a Michael addition. This reaction yields the PEG amino acrylate 50, which may then be utilized to prepare the prepolymerization solution 100 (shown in FIG. 2).

In one embodiment, the prepolymerization solution 100 may be generated by adding 1.2 mL triethanolamine (TEOA) 110 and 260 μL of 6 N HCl to 39.4 mL of 10 mM HEPES buffered saline (HBS) at pH 7.4. In this embodiment, PEG-diacrylate (230 mg) 30 is dissolved in 770 μL of 10 mM HBS, and 115 mM TEOA buffer 110, pH 8. Additionally, 15 μL of 235.3 mM cysteamine 20 is added to 115 μL of 1 mM MES buffered saline and then added to 870 μL of the PEG-diacrylate 30 solution, which is prepared with 25% PEG diacrylate 30. This solution is incubated for 10 minutes in the dark at room temperature 40. 11.8 μL of 1-vinyl 2-pyrrolidinone (NVP) 120 is added to this mixture to yield the prepolymerization solution 100. It will be appreciated, however, that this is merely one method of forming the prepolymerization solution, and that other methods and mixtures may be utilized. The prepolymerization solution 100 is thereafter used to form the hydrogel layers.

FIG. 2 demonstrates one method of formation of the hydrogel multilayers. According to an embodiment, the prepolymerization solution generated as shown in FIG. 1 is attached to a solid support. In the embodiment shown in FIG. 2, the solid support is a glass slide 60. In another possible embodiment (not shown), the support may be a self-standing, thin hydrogel membrane. It will be appreciated, however, that any suitable support may be utilized.

In the embodiment, the glass slide 60 is derivatized with eosin and Woodward's reagent solution 80 to yield an eosin derivatized surface 90 containing an eosin molecule 160. The prepolymerization solution may then be placed onto the eosin derivatized surface 90. Modified PEG is used for the first layer 140 in order to facilitate the attachment of eosin 160 to the solution. The modified PEG is polymerized via a free-radical mechanism 135 to form the first hydrogel layer 140. In an embodiment, the free radical mechanism reaction is generated through illumination with a laser, such as an argon ion laser. In an embodiment, the wavelength of the argon ion laser is approximately 514 nanometers. It will be appreciated that nanometers is commonly abbreviated as “n.”

It will be appreciated that any acceptable illumination mechanism may be utilized. It will further be appreciated that any necessary wavelength may likewise be utilized. For example, one embodiment uses laser light 130 with a longer wavelength than ultraviolet light, which is less damaging to biological material. Wavelengths in the visible portion of the electromagnetic spectrum are less likely to cause mutations or other problems related to the breaking of chemical bonds.

A subsequent layer of hydrogel 170 may thereafter be formed on top of the first layer. In this embodiment, the glass slide 60 with the first layer of hydrogel 140 is immersed in eosin and Woodward's reagent solution 80. In an embodiment, the slide is immersed for five minutes, in order to allow reaction of amine groups 150 present in the PEG molecule 50 with the carboxyl groups 155 present in the eosin molecule 160.

The slide 60 with the first hydrogel layer 140 is then rinsed off and PEG diacrylate 30 precursor solution 190 is placed onto the surface of the first layer. In an embodiment, 20 μL of the PEG diacrylate 30 is used. The slide 60 is thereafter illuminated with an argon ion laser for 2 minutes 130.

In another embodiment, attachment of the first hydrogel layer 140 to the surface of the solid substrate can be accomplished by exposing the eosin functionalized substrates 160 to a mixture of hydrogel precursors (such as monomers, macromers, oligomers and electron donors). Upon activation with visible light, these hydrogel precursors are initiated.

In an embodiment, the outermost hydrogel layer can be formed through photopolymerization of PEG diacrylate 20 solution 190 only, without any PEG modification. This can be accomplished because no further covalent boding of the photoinitiator to the surface is necessary.

Formation of multiple layers of hydrogel could significantly alter the properties of the hydrogel system itself, such as where each layer has its own properties. For example, the permeability of each layer could be different, or each layer could be impermeable to molecules of differing sizes. As a result, a hydrogel containing multiple layers will have significant weight increase from a single-layer hydrogel. This is illustrated in TABLE 1.

TABLE 1
Total Weight Increase of the Glass Slide

| Number of Layers Added | Weight Increase (mg) |
|---|---|
| 1 | 6.4 |
| 2 | 11.0 |
| 3 | 16.4 |

TABLE 1 depicts gravimetric measurements of the surface of a glass slide 60 after polymerization. As shown in the table, following polymerization of the first layer an increase of 6.4 mg of the total weight of the glass slide 60 was seen. Increases in weight after the formation of the second 170 and third layers 180 of hydrogel were measured as 11.0 and 16.4 mg respectively.

In further embodiments, various monomers or macromers, or both, may be incorporated into the hydrogels during the formation of individual layers. Such a feature can affect the structure or function or both of the resulting hydrogel.

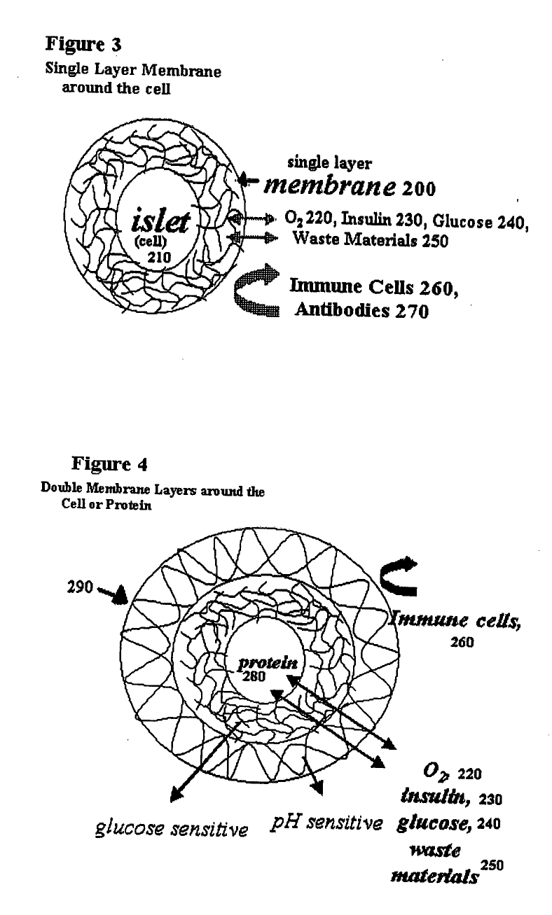

As illustrated in FIGS. 3 and 4, the presence of a multilayer hydrogel presents significant benefits during use. FIG. 3 is a graphic representation of an embodiment of a traditional single layer membrane 200 around an islet cell 210. The single layer membrane 200 enables the exchange of oxygen 220, insulin 230, glucose 240 and/or waste materials 250 through the single layer membrane 200, while preventing the exchange of immune cells 260 and antibodies 270 through the single layer membrane 200.

In comparison, FIG. 4 is a graphic representation of an embodiment of a double layer membrane 290 around a cell or islet 210. As shown in this embodiment, an advantage of a double membrane 290 is that each layer can have different properties. For example, one layer can accomplish the function of immunoprotection by preventing the exchange of immune cells 260 and antibodies 270 while allowing the passage of nutrients (such as glucose 240), oxygen (220) and insulin (230). The second layer, however, may have sensing and actuating functions. For example, the permeability can change in response to local changes in pH or glucose concentration 300.

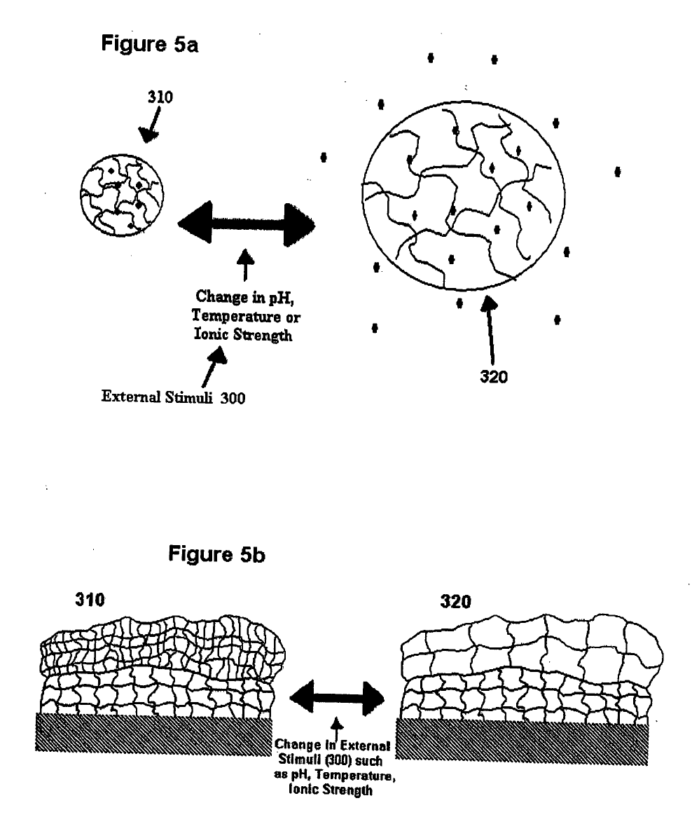

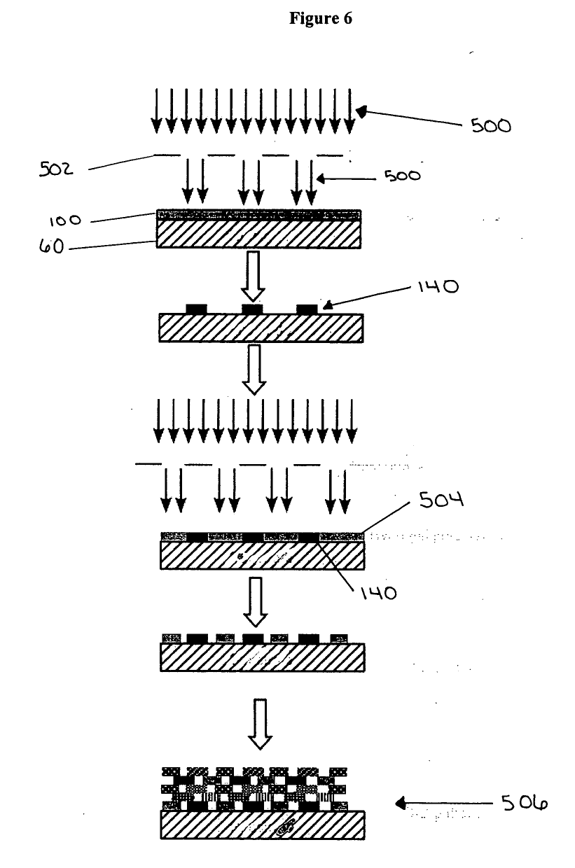

Additionally, the multilayer hydrogels of the present invention may be utilized in a number of different functions. As discussed above, in an embodiment, shown in FIG. 4, hydrogels surround cells or proteins to facilitate entry into and exit from the cell. In another embodiment, FIG. 5a and FIG. 5b demonstrate hydrogel response to external stimuli. The figures demonstrate a responsive multilayer hydrogel 310 before an external stimulus 300 and a multilayer hydrogel 320 after an external stimulus 300. In these embodiments, the outer layer of the hydrogel changes permeability in response to an external stimuli 300.

In another embodiment, the multilayer hydrogels incorporate other elements into the layers, to aid in functionality. Such elements may include, but are not limited to, proteins, enzymes, or fluorescent molecules. For example, specific enzymes may be incorporated such that, in the presence of specific substrates, the products of the enzymatic reaction could change the pH within that hydrogel 320 and thus affect the physicochemical and transport properties. These multilayers 310, 320 could be used to trigger the release of specific substances incorporated within the hydrogel 310 and thus, be used in programmed or controlled drug release.

In another embodiment, the method of forming a multilayer hydrogel 310 is implemented for immunosuppression of transplants through cell encapsulation. In this embodiment, a hydrogel barrier is used to prevent the rejection of transplanted cells by protecting the cells from a host's immune system, while enabling the transmission of other molecules necessary for the cells' survival.

For example, in an embodiment a hydrogel barrier may be utilized to protect transplanted tissue or organs from the host's immune system while another (external) layer can incorporate extracellular matrix molecules that promote cell adhesion. Angiogenesis promoting molecules such as vascular endothelial growth factor (VEGF) are likewise incorporated in such a way that the implant can become vascularized. The formed vasculature would nourish the encapsulated cells and the hydrogel barrier would prevent the implanted cells from being destroyed by the immune system. Using the hydrogel to avoid rejection by the immune system additionally reduces the need for immunosuppressive drugs.

In another embodiment, the hydrogel layers 310 may be used to encapsulate a drug delivery device, such as a pill or implant. Hydrogel layers 310 could control the release of molecules into the user's body over time, or in response to the external conditions 300. Such embodiments lower dosage requirements or avoid the need to continuously administer medication over time. In an embodiment, hydrogel layers 310 could lower the cost of medication or maintain a more consistent amount of medication in the host, enabling more effective treatments with fewer side effects.

In an embodiment, the formation of multifunctional biosensors with three-dimensional patterning of sensing molecules encapsulated within the hydrogels 310 enables high densities of sensing elements in a biochip. Sensing molecules could include, for example, antibodies, enzymes or nucleic acids, enabling detection of many substances at once, even in small quantities.

An embodiment includes multilayered membranes 310 with sensing and responsive properties (smart hydrogels). This embodiment enables the creation of smart sensing materials that respond to external stimuli 300 and generate signals depending on the environment. Responsive hydrogels reduce the amount of medical attention needed after treatment. In an embodiment, hydrogel multilayers signal changes in physiological conditions during diagnosis or monitoring, thereby reducing medical costs through earlier intervention.

Actuating smart polymers incorporated within some layers in an embodiment decrease or increase their permeability. An embodiment allows or restricts the passage of molecules depending on environmental conditions 300 (a smart membrane with sensing properties). Hydrogel layers 310 enabling environmental conditions to dictate the qualities or response of the layers in an embodiment reduces the need for treatment, for example by releasing molecules or forming a barrier.

In an embodiment, hydrogel multilayers 310 with variable permeability are manipulated to control the amount of drug therapy necessary, by enabling increased passage of molecules during drug therapy. The layers may block the amount of molecules permeating the hydrogel during drug therapy.

In an embodiment, formation of PEG diacrylate 30 hydrogel patterns by using microcontact printing on initiator-immobilized glass surfaces is used for incorporating biosensor elements into microelectronic and micro-optical devices. The hydrogel layers 310 then provide a hospitable environment, with water and other molecules available, or structural support for stabilizing the biosensor elements.

In yet another embodiment, hydrogel multilayers with varying permeability can be used as gradient separation membranes. Larger molecules become blocked or trapped within large pores of the first hydrogel layers while smaller molecules are blocked or trapped within smaller pores of lower, more crosslinked hydrogel layers. This gradual separation of molecules according to molecular size can be used to purify molecules of widely dissimilar molecular dimensions that are present in small quantities in a complex sample such as a biological fluid.

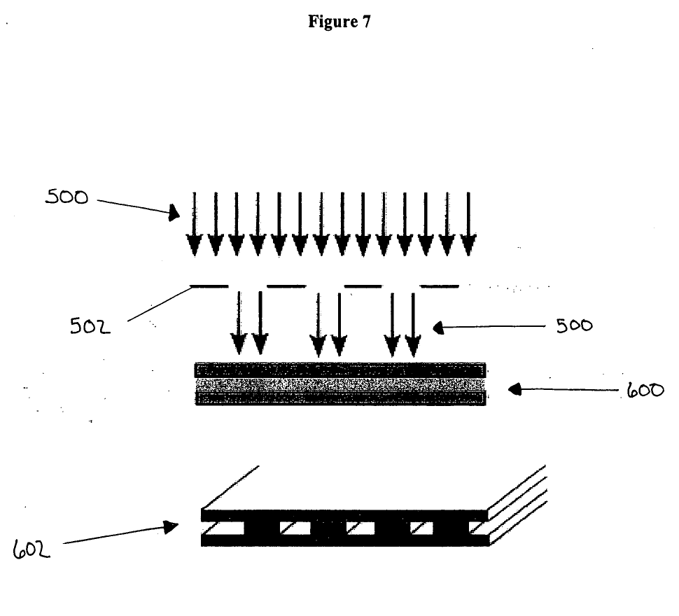

In further embodiments, illustrated in FIGS. 6 and 7, multilayered hydrogels may be formed having multi-dimensional patterns and/or regions made of differing compositions. In these embodiments, only selected areas of the surface of the selected hydrogel layers are exposed to the radiation. As shown in FIG. 6, for example, a photomask 502 is interposed between the substrate 60 having a first layer of prepolymerization solution 100 and the laser illumination source (not shown), which emits radiation waves 500. In this example, only those regions on the exposed surface 100 will polymerize to form the first hydrogel layer 140. As the unexposed region does not polymerize, it can be rinsed off of the surface. This procedure may be repeated many times to add regions 504 and to produce complex features on the hydrogel surface.

Where an even more complex composition is required, as illustrated generally at FIG. 7, the patterns may be implemented on each of the different layers so that vertical composition can be controlled. As illustrated in the figure, the radiation waves 500 are again directed by using a photomask 502. The radiation is directed onto the surface of multiple layers of hydrogel 600. In this regard, this method can be used to form a multilayer hydrogel 602 having microchannels or microcavities within the hydrogel system, providing added function of the hydrogel.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A multiple-layer hydrogel comprising:
(a) a first layer comprising polyethylene glycol; and
(b) at least one subsequent layer comprising polyethylene, wherein the at least one subsequent layer is covalently attached to the first layer.

2. The multiple-layer hydrogel of claim 1 wherein the first layer has been exposed to a photoinitiator.

3. The multiple-layer hydrogel of claim 1 further comprising one or more enzymes.

4. The multiple-layer hydrogel of claim 1 wherein the hydrogel is responsive to external stimuli.

5. The multiple-layer hydrogel of claim 4 wherein the external stimuli include pH.

6. The multiple-layer hydrogel of claim 1 wherein at least one layer forms a pattern.

7. The multiple-layer hydrogel of claim 6 wherein the pattern is three-dimensional.

8. A method of forming a hydrogel comprising:
(a) preparing a solution;
(b) applying a first layer of the solution to a support surface;
(c) exposing the first layer of solution to radiation; and
(d) applying a second layer of the solution to the first layer.

9. The method of claim 8 further comprising the steps of:
(e) exposing the second layer to radiation; and
(f) applying a third layer of the solution to the second layer.

10. The method of claim 9, further comprising repeating steps (e) to (f) to form subsequent layers.

11. The method of claim 9 wherein the radiation has a wavelength of about 514 nm.

12. The method of claim 8, wherein the solution is a prepolymerization solution.

13. The method of claim 12, wherein the prepolymerization solution comprises polyethylene glycol.

14. A method of forming a hydrogel, the method comprising the steps of:
(a) mixing a prepolymerization solution;
(b) providing a support surface and exposing the surface to a photoinitiator;
(c) depositing a first layer of the prepolymerization solution onto the surface;
(d) exposing the first layer to radiation;
(e) exposing the first layer to the photoinitiator;
(f) depositing a second layer of prepolymerization solution onto the first layer; and
(g) exposing the second layer to radiation.

15. The method of claim 14, further comprising repeating steps (e) to (g) to form subsequent layers.

16. The method of claim 14 wherein the radiation has a wavelength of about 514 nm.

17. The method of claim 14 wherein the prepolymerization solution includes at least one macromer.

18. The method of claim 14 wherein the prepolymerization solution includes at least one monomer.

19. The method of claim 14 wherein the prepolymerization solution comprises polyethylene glycol.

20. The method of claim 19 wherein the prepolymerization solution is prepared using polyethylene glycol amino acrylate.
